(19) United States
(12) Patent Application Publication
(10) Pub. No.: US 2002/0037281 A1
(43) Pub. Date: Mar. 28, 2002

(54) Methods of transducing neural cells using lentivirus vectors

(76) Inventors: Beverly L. Davidson, North Liberty, IA (US); Joseph M. Alisky, Iowa City, IA (US); Thomas W. Dubensky JR., Piedmont, CA (US); Stephanie M. Hughes, Iowa City, IA (US); Douglas Jolly, Encinitas, CA (US); Sybille L. Sauter, Del Mar, CA (US)

Correspondence Address: CHIRON CORPORATION, Intellectual Property - R440, P.O. Box 8097, Emeryville, CA, 94662-8097, US

(21) Appl. No.: 09/866,532

(22) Filed: May 25, 2001

Related U.S. Application Data
(60) Provisional application No. 60/207,541, filed on May 26, 2000.
(60) Provisional application No. 60/279,035, filed on Mar. 27, 2001.

(51) Int. Cl. C12N 15/867; A61K 48/00; C12N 5/08

(52) U.S. Cl. 424/93.21; 435/456; 435/368

(57) ABSTRACT

Gene delivery vectors, such as, for example, recombinant FIV vectors, and methods of using such vectors are provided for use in transducing neural cells, such as neural progenitor cells, and cerebellar neurons, in particular, Purkinje cells.

### CROSS-REFERENCE TO RELATED APPLICATION

This application is related to provisional patent application Ser. No. 60/207,541, filed May 26, 2000, and provisional patent application Ser. No. 60/279,035, filed Mar. 27, 2001, from which applications priority is claimed under 35 USC § 119(e)(1) and which applications are incorporated herein by reference in their entireties.

### TECHNICAL FIELD

The present invention relates to methods for transducing neural cells and methods for treating diseases of the central nervous system. In particular, the present invention pertains to the use of various gene delivery vectors which direct the expression of selected gene products in neural progenitor cells and/or cerebellar neurons.

### BACKGROUND OF THE INVENTION

Degenerative diseases of the cerebellum are potentially amenable to gene therapy if transduction to sufficient numbers of neurons can be achieved. For example, some of the autosomal dominant spinocerebellar ataxias (SCA) are due to loss of Purkinje cells, inferior olivary and pontine neurons, and to a lesser extent granule cells (Kato et al., Acta Neuropathol 96:67-74, 1998; Koeppen, A. H., J. Neuropath and Experimental Neurology 57:531-543, 1998). Onset is typically from the fifth to seventh decade of life, with degeneration occurring over a decade. The underlying genetic defect in several types of the SCA (SCA-1, SCA-2, SCA-3, SCA-6, and SCA-7) causes polyglutamine tract expansion and a toxic gain of function in the encoded protein (Klockgether and Evert, Trends Neurosci 21:413-418, 1998; Paulson et al., Am J. Hum Genet 64:339-345, 1999.

Neural progenitor or stem cells are a potential target for neurodegenerative disease therapy. Progenitor cells may be used to replace neural cell types, neurons, astrocytes or oligodendrocytes and to act as a vector for delivery of therapeutic molecules to the degenerating CNS. Delivery of therapeutic gene products or differentiation cues would be enhanced by viral-mediated gene delivery however, the effects of viral-mediated gene transfer on primary neural progenitor cells have not heretofore been well characterized.

Neural progenitor cells are found in the CNS throughout life. In the adult, neurogenesis is maintained in the ventricular region (Altman, J., J. Comp.Neurol. 137:433-458, 1969; Corotto et al., Neurosci.Lett. 149:111-114, 1993) where precursor cells migrate through the rostral migratory stream to the olfactory bulb, differentiating into granule and periglomerular neurons (Altman, J., J. Comp.Neurol. 137:433-458, 1969; Lois et al., Science 264:1145-11483, 1994). Granule neurons also continue to differentiate from progenitors in the subgranular zone of the dentate gyrus (Altman et al., J. Comp.Neurol. 124:319-336, 1965).

Neural progenitor cells can be isolated from either embryonic or adult brain and maintained in culture either as primary cultures or immortalized cell lines (Reynolds et al. Science 255:1707-1710, 1992; Reynolds et al., J. Neurosci. 12:4565-4574, 1992; Snyder et al., Cell 68:33-51, 1992). In primary culture, nestin-positive progenitor cells are cultured in the presence of epidermal and/or basic fibroblast growth factors generating clones of progenitor cells commonly referred to as neurospheres (Reynolds et al. Science 255:1707-1710, 1992; Gage et al., PNAS. 92:11879-11883, 1995), which with removal of mitogens, will differentiate into all major neural cell types (Reynolds et al. Science 255:1707-1710, 1992; Reynolds et al., J. Neurosci. 12:4565-4574, 1992).

Progenitor cells immortalized by retroviral-mediated v-myc gene transfer have been used for cell replacement and delivery of secreted gene products. Over expression of Nurr-1, required for induction of dopaminergic phenotype, in the murine cerebellar progenitor cell line C17.2 (Snyder et al., Cell 68:33-51, 1992) resulted in differentiation of progenitors into TH-positive neurons in the presence of type-I astrocytes (Wagner et al., Nat Biotechnol 17:653-659, 1999). The delivery of β-glucuronidase to newborn MPS VII mice using modified C17.2 cells showed that these cells can secrete gene products and correct lysosomal pathology (Snyder et al., Nature 374:367-370, 1995). Migration of implanted immortalized or primary neural progenitor cells is enhanced in the setting of CNS injury. For example, progenitor cells migrate towards tumors (Benedetti et al., Nat.Med. 6:447-450, 2000; Herrlinger et al., Mol. Ther. 1:347-357) or apoptotic areas (Snyder et al., PNAS 94:11663-11668, 1997).

Previous investigations of gene transfer in the cerebellum has examined replication-deficient adenovirus vectors (rAd). Adenoviruses are non-integrating, non-enveloped DNA viruses. rAd expressing β-galactosidase injected into the cerebellar cortex of mice transduced numerous precerebellar neurons in the brainstem (Terashima et al., Anat Embryol 196:363-382, 1997). This occurred via retrograde axonal transport of virions from mossy fiber terminals in the cortex back to neuronal soma. However, within the cortex itself mainly glia were transduced, with only minimal transfection of Purkinje cells or other classes of neurons. Viral vectors that transduce cerebellar neurons would be preferable in the study of the spinal cerebellar ataxias, and for testing therapies in representative animal models (Vig et al., J. Neurol Sci 174:100-110, 2000; Lorenzetti et al., Hum Mol Genet 9:779-785, 2000).

Recombinant adenoassociated viruses (rAAV) have been shown to mediate gene transfer to neurons when injected into the rodent cerebrum (During et al., Gene Therapy 5:820-827, 1998; Davidson et al., PNAS 97:3428-3432, 2000). AAVs are DNA dependoviruses, and require adenovirus or herpesvirus as helper for productive infections. Earlier studies showed that vectors derived from AAV2 efficiently transduce neurons immediate to the site of administration in the hippocampus and inferior colliculi of rats (Bartlett et al., Hum Gene Ther 9:1181-1186, 1998; Davidson et al., PNAS97:3428-3432, 2000). More recently, rAAV5-based vectors have been shown to be capable of diffusion within the mouse striatum well beyond the injection site (Davidson et al., PNAS 97:3428-3432, 2000). Similar to rAAV2 vectors, rAAV5 vectors predominantly transduced neurons in the hippocampus, cortex, striatum or medial septum.

Recombinant retroviral gene delivery methods have been extensively utilized in gene therapy approaches, in part due to: (1) the efficient entry of genetic material (the vector genome) into cells; (2) an active, efficient process of entry into the target cell nucleus; (3) relatively high levels of gene expression; (4) the potential to target particular cellular subtypes through control of the vector-target cell binding and the tissue-specific control of gene expression; (5) a general lack of pre-existing host immunity; (6) substantial knowledge and clinical experience which has been gained with such vectors; and (7) the capacity for stable and long-term expression.

Briefly, retroviruses are diploid positive-strand RNA viruses that replicate through an integrated DNA intermediate. Upon infection by the RNA virus, the retroviral genome is reverse-transcribed into DNA by a virally encoded reverse transcriptase that is carried as a protein in each retrovirus. The viral DNA is then integrated pseudo-randomly into the host cell genome of the infected cell, forming a “provirus” which is inherited by daughter cells.

One type of retrovirus, the murine leukemia virus, or “MLV”, has been widely utilized for gene therapy applications (see generally Mann et al. Cell 33:153, 1983; Cane and Mulligan, PNAS 81:6349, 1984; and Miller et al., Human Gene Therapy 1:5-14, 1990). One major disadvantage of MLV-based vectors, however, is that the host range (i.e., cells infected with the vector) is limited, and the frequency of transduction of non-replicating cells is generally low.

Adenovirus and AAV, as well as lentivirus, can infect terminally differentiated cells without the need for cell division, and have thus been used for gene transfer to the CNS where cell division is limited (Davidson et al., Nat. Genet. 3:219-223, 1993; Mastrangeli et al., Clin.Res. 41:223A(Abstract), 1993; Ghadge et al., Gene Ther. 2:132-137, 1995; Xiao et al., Exp.Neurol. 144:113-124, 1997; McCown et al., Brain Res. 713:99-107, 1996; Chamberlin et al., Brain Res. 793:169-175, 1998; Blomer et al., J. Virol. 71:6641-6649, 1997; Zufferey et al., Nat Biotechnol 15:871-875, 1997; Kordower et al., Exp.Neurol. 160:1-16, 1999). Feline immunodeficiency virus (“FIV”) is an RNA virus of the lentivirus family that infects both dividing and non-dividing cells and integrates into the host genome, allowing transgene maintenance in dividing cells. FIV-mediated gene therapy vector systems have also been described (see, International Publication Nos. WO 99/15641 and WO 99/36511).

Replication incompetent recombinant lentiviral vectors derived from human immunodeficiency virus (rHIV) and rFIV show tropism for neurons in vitro (Poeschla et al., Nat Med 4:354-357, 1998) and in vivo when injected into the cerebrum (Science 272:263-267, 1996; Naldini, J. Virol, 1996). The recombinant lentivirus vectors remain capable of infecting non-dividing cells when deleted of accessory proteins (Johnston et al., J. Virol 73:4991-5000, 1999, Naldini, Throm Haemat 82:552-554, 1999). In recent studies, rHIV vectors pseudotyped with the vesicular stomatitus glycoprotein G (VSV-g) envelope protein mediated gene transfer to a large number of striatal neurons when injected into non-human primate brain, with no apparent decline in transgene expression throughout the three month study (Kordower et al., Exp Neurol 160:1-16, 1999). Similar results have been found with lentivirus vectors based on FIV. Such vectors have also been shown to be effective in infecting hematopoietic stem cells. (An et al., J. Virol. 74:1286-1295, 2000; Sutton et al., J. Virol. 73:3649-3660, 1999; Uchida et al., PNAS 95:11939-11944, 1998; Case et al., PNAS96:2988-2993, 1999; Miyoshi et al., Science 283:682-686, 1999; Evans et al., Hum. Gene Ther. 19:1479-1489, 1999). However, lentiviral infection of primary neural progenitor cells has not previously been reported.

The present invention provides methods for transducing neural progenitor cells and cerebellar neurons, as well as methods for treating and preventing a number of diseases associated with the central nervous system and cerebellar degeneration, using retrovirus-mediated gene transfer and, further, provides other related advantages.

### SUMMARY OF THE INVENTION

The present invention provides methods for transducing neural cells, including neural progenitor cells and cerebellar neurons. The methods are useful for studying CNS and cerebellar disorders, and for testing therapies in representative animal models. The invention also provides methods for treating, preventing, or inhibiting diseases of the brain and other disorders of the central nervous system (CNS), such as but not limited to, Parkinson's, multiple sclerosis, Alzheimer's, and other diseases that cause cerebellar degeneration. Transduced progenitor cells may be used to replace neural cell types, neurons, astrocytes and/or oligodendrocytes and therefore to deliver therapeutic molecules to degenerating CNS.

In particular, it has been surprisingly found that cerebellar neurons and neural progenitor cells can be effectively transduced using FIV vectors. Nestin-positive neurospheres can be regenerated from single FIV-infected progenitors, indicating that FIV infection does not inhibit progenitor cell self-renewal. FIV-infected progenitors also retain the potential for differentiation, such as into neurons and glia.

Thus, within one aspect of the present invention, methods are provided for treating or preventing diseases of the CNS and/or cerebellum comprising the step of direct introduction to the CNS or cerebellum a gene delivery vector which directs the expression of one or more polypeptides, proteins or enzymes, such that the disease is treated or prevented. Within certain embodiments of the invention, a viral promoter (e.g., CMV), a tissue-specific promoter (e.g., opsin, RPE, cholecystokinin (see, U.S. Pat. No. 5,681,744) and neuropeptide Y promoter), or an inducible promoter (e.g., tet) is utilized to drive the expression of the polypeptide, protein or enzyme factor.

According to a preferred embodiment, the invention provides a method of transducing neural progenitor cells, or Purkinje cells of the cerebellum.

Preferred gene delivery vectors suitable for use with the present invention may be generated from retroviruses such as FIV or HIV. In a particularly preferred embodiment, the gene delivery vector is an FIV vector.

Utilizing the methods and gene delivery vectors provided herein a wide variety of CNS and cerebellar diseases and disorders may be readily treated or prevented, including for example, spinocerebellar ataxias (SCA) such as SCA-1, SCA-2, SCA-3, SCA-6, and SCA-7; cerebellar degeneration due to alcoholism; idiopathic Purkinje cell degeneration; lithium intoxication; ceroid lipofuscinosis; ataxia telangiectasia; high dose arabinoside; Huntington's disease; fragile X syndrome; hereditary motor and sensory neuropathy and cerebellar atrophy; Alzheimer's disease (both sporadic and familial); normal aging; Parkinson's Disease and Parkinson's disease-like symptoms such as muscle tremors, muscle weakness, rigidity, bradykinesia, alterations in posture and equilibrium and dementia; demyelinating diseases such as, but not limited to, multiple sclerosis, parainfectious disorders such as acute disseminated encephalomyelitis and acute hemorrhagic leukoencephalopathy, viral infections such as progressive multifocal leukoencephalopathy and subacute sclerosing panencephalitis, nutritional disorders such as vitamin B₁₂ deficiency, demyelination of the corpus callosum (Marchiafava-Bignami disease) and central pontine myelinolysis, anoxic-ischemic sequelae such as delayed postanoxic cerebral demyelination and progressive subcortical ischemic encephalopathy; dismyelinating diseases such as, but not limited to, the leukocystrophies such as metachromatic leukodystropy, sudanophilic (Pelizaeus-Merzbacher disease), globoid cell (Krabbe's disease), adrenoleukodystropy (Schilder's disease), Alexander's disease, Canavan's disease, Seitelberger's disease, aminoaciduias; multisystem atrophy; paraneoplastic Purkinje cell degeneration; metachromatic leukodystrophy (enzymedeficient and activator-deficient form); manic depression; bipolar disorders; schizophrenia; autism; traumatic brain and spinal cord injury, and the like.

Accordingly, the methods of the present invention may be used to alleviate abnormalities of the CNS and cerebellum that result in demyelination, dysmyelination, dementia, dysmetria, ataxia, past pointing, dysdiadochokinesia, dysarthria, intention and action tremor, cerebellar nystagmus, rebound, hypotonia, and loss of equilibrium.

Genes encoding a wide variety of polypeptides, proteins or enzymes may be employed, including those which, when expressed, prevent or alleviate the effects of the particular CNS and/or cerebellar disorder in question. Examples of such proteins include, but are not limited to CLN2 (tripeptidyl protease; ttp); CLN3; CLN1 (protein palmitoyl thioesterase); calbindin; glutamate decarboxylase; the genes encoding proteins deficient in SCA-1, SCA-2, SCA-3, SCA-6, and SCA-7; ataxin (1-7); arylsulfatase A, sulfatide activator/saposin; galactosylceramidase; various growth factors such as any of the various NGFs and FGFs, as well as CNTF, BDNF, GDNF, NT3, NT4/5, and IGF-1; monoamine oxidase; tyrosine hydroxylase; the Huntington (htt) gene; bipolar genes such as G-protein alpha subunit gene and Galphaz (GNAZ); serotonin transporter gene; serotonin receptor HTR-7, genes in the VCSF region of chromosome 22; and antiapoptotic genes. Moreover, critical enzymes involved in the synthesis of neurotransmitters such as dopamine, norepinephrine, and GABA have been cloned and available and can be used to treat a broad range of brain disease in which disturbed neurotransmitter function plays a crucial role, such as schizophrenia, manic-depressive illnesses and Parkinson's Disease. It is well established that patients with Parkinson's suffer from progressively disabled motor control due to the lack of dopamine synthesis within the basal ganglia. The rate limiting step for dopamine synthesis is the conversion of tyrosine to L-DOPA by the enzyme, tyrosine hydroxylase. L-DOPA is then converted to dopamine by the ubiquitous enzyme, DOPA decarboxylase. Thus, the genes for tyrosine hydroxylase and DOPA decarboxylase can be delivered by the techniques described herein in order to treat such diseases as Parkinson's. In addition, the enzymes responsible for neurotransmitter synthesis can be delivered using the systems described herein. For example, the gene for choline acetyl transferase may be expressed within the brain cells (neurons or glial) of specific areas to increase acetylcholine levels and improve brain function. For treating multiple sclerosis, the genes encoding MPIF-1, MIP-4, M-CIF and anti-inflammatory proteins can be delivered (see, U.S. Pat. No. 6,001,606).

These and other aspects of the present invention will become evident upon reference to the following detailed description and attached drawings. In addition, various references are set forth herein which describe in more detail certain procedures or compositions, and are therefore incorporated by reference in their entirety.

### DETAILED DESCRIPTION OF THE INVENTION

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990).

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “a gene” includes a mixture of two or more genes, and the like.

### Definitions

In describing the present invention, the following terms will be employed, and are intended to be defined as indicated below.

“Gene delivery vehicle” refers to a construct which is capable of delivering, and, within preferred embodiments expressing, one or more gene(s) or sequence(s) of interest in a host cell. Representative examples of such vehicles include viral vectors, nucleic acid expression vectors, naked DNA, and certain eukaryotic cells (e.g., producer cells).

The terms “lentiviral vector construct,” “lentiviral vector,” and “recombinant lentiviral vector” are used interchangeably herein and refer to a nucleic acid construct derived from a lentivirus which carries, and within certain embodiments, is capable of directing the expression of a nucleic acid molecule of interest. Lentiviral vectors can have one or more of the lentiviral wild-type genes deleted in whole or part, as described further below, but retain functional flanking long-terminal repeat (LTR) sequences (also described below). Functional LTR sequences are necessary for the rescue, replication and packaging of the lentiviral virion. Thus, a lentiviral vector is defined herein to include at least those sequences required in cis for replication and packaging (e.g., functional LTRs) of the virus. The LTRs need not be the wild-type nucleotide sequences, and may be altered, e.g., by the insertion, deletion or substitution of nucleotides, so long as the sequences provide for functional rescue, replication and packaging.

Generally, a lentiviral vector includes at least one transcriptional promoter or promoter/enhancer or locus defining element(s), or other elements that control gene expression by other means such as alternate splicing, RNA export, post-translational modification of messenger, or post-transcriptional modification of protein. As explained above, such vector constructs also include a packaging signal, LTRs or functional portions thereof, and positive and negative strand primer binding sites appropriate to the retrovirus used (if these are not already present in the retroviral vector). Optionally, the recombinant lentiviral vector may also include a signal that directs polyadenylation, selectable and/or non-selectable markers, an origin of second strand DNA synthesis, as well as one or more restriction sites and a translation termination sequence. Examples of markers include, but are not limited to, neomycin (Neo), thymidine kinase (TK), hygromycin, phleomycin, puromycin, histidinol, green fluorescent protein (GFP), human placental alkaline phosphatase (PLAP), DHFR, β-galactosidase and human growth hormone (hGH). By way of example, such vectors typically include a 5′ LTR, a tRNA binding site, a packaging signal, an origin of second strand DNA synthesis, and a 3′ LTR or a portion thereof.

The terms “FIV retroviral vector construct,” “FIV vector,” and “recombinant FIV vector” are used interchangeably to refer to a lentiviral vector construct, as defined above, which includes one or more FIV sequences. By way of example, such vectors typically include a 5′ FIV LTR, a primer binding site, a packaging signal, an origin of second strand DNA synthesis, and a 3′ FIV LTR. Heterologous sequences that are included in the vector construct are those which encode a protein, such as an enzyme, the expression of which is deficient in the selected target cells.

“Expression cassette” refers to an assembly which is capable of directing the expression of the sequence(s) or gene(s) of interest. The expression cassette includes a promoter or promoter/enhancer which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include a bacterial origin of replication, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

“Packaging cell” refers to a cell which contains those elements necessary for production of infectious recombinant retrovirus which are lacking in a recombinant retroviral vector. Packaging cells contain one or more expression cassettes which are capable of expressing proteins which encode gag, pol and env-derived proteins. Packaging cells can also contain expression cassettes encoding one or more of vif rev, or ORF 2 in addition to gag/pol and env expression cassettes.

“Producer cell” and “Vector Producing Cell Line” (VCL) refer to a cell which contains all elements necessary for production of recombinant vector particles.

“Lentiviral vector particle” as used herein refers to a recombinant lentivirus which carries at least one gene or nucleotide sequence of interest, which is generally flanked by lentiviral LTRs. The lentivirus may also contain a selectable marker. The recombinant lentivirus is capable of reverse transcribing its genetic material into DNA and incorporating this genetic material into a host cell's DNA upon infection. Lentiviral vector particles may have a lentiviral envelope, a non-lentiviral envelope (e.g., an amphotropic or VSV-G envelope), a chimeric envelope or a modified envelope (e.g., truncated envelopes or envelopes containing hybrid sequences).

“FIV vector particle” as utilized herein refers to a lentiviral particle, as defined above, which is derived from FIV.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. Gene 13:197, 1981. Such techniques can be used to introduce one or more exogenous DNA moieties, such as a plasmid vector and other nucleic acid molecules, into suitable host cells. The term refers to both stable and transient uptake of the genetic material.

The term “transduction” denotes the delivery of a DNA molecule to a recipient cell either in vivo or in vitro, via a replication-defective viral vector, such as via a recombinant lentiviral vector particle.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “5,” or “3” relative to another sequence, it is to be understood that it is the position of the sequences in the non-transcribed strand of a DNA molecule that is being referred to as is conventional in the art.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

By “vertebrate subject” is meant any member of the subphylum chordata, including, without limitation, mammals such as cattle, sheep, pigs, goats, horses, and human and non-human primates; domestic animals such as dogs and cats; laboratory animals including rodents such as mice, rats and guinea pigs, and the like; birds, including domestic, wild and game birds such as cocks and hens including chickens, turkeys and other gallinaceous birds; and fish. The term does not denote a particular age. Thus, both adult and newborn animals, as well as fetuses, are intended to be covered.

### MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

As noted above, the present invention provides methods for treating, preventing, or, inhibiting diseases of the CNS and brain, comprising the general step of administering a recombinant lentiviral vector which directs the expression of one or more polypeptides, proteins or enzymes, such that the disease is treated or prevented. The invention is also directed to transducing neural progenitor cells and cerebellar neurons, such as Purkinje cells. The invention is based on the surprising finding that FIV-based vectors can infect cerebellar neurons, as well as progenitor cell populations. Moreover, infection does not inhibit the ability of neural progenitor cells to differentiate into multiple cell types, or to respond to injury within the CNS. Thus, the present invention provides for the use of genetically-manipulated stem cells for CNS therapies.

In order to further an understanding of the invention, a more detailed discussion is provided below regarding (A) gene delivery vectors; (B) polypeptides, proteins or enzymes for use in treating cerebellar diseases; and (C) methods of administering the gene delivery vectors in the treatment or prevention of these diseases.

A. Gene Delivery Vectors

1. Construction of Retroviral Gene Delivery Vectors

Within one aspect of the present invention, retroviral gene delivery vehicles are provided which are constructed to carry or express a selected gene(s) or sequence(s) of interest. Briefly, retroviral gene delivery vehicles of the present invention may be readily constructed from a wide variety of retroviruses, including for example, B, C, and D type retroviruses as well as spumaviruses and lentiviruses such as FIV, HIV, HIV-1, HIV-2 and SIV (see RNA Tumor Viruses, Second Edition, Cold Spring Harbor Laboratory, 1985). Such retroviruses may be readily obtained from depositories or collections such as the American Type Culture Collection (“ATCC”; 10801 University Blvd., Manassas, Va. 20110-2209), or isolated from known sources using commonly available techniques.

Any of the above retroviruses may be readily utilized in order to assemble or construct retroviral gene delivery vehicles given the disclosure provided herein, and standard recombinant techniques (e.g., Sambrook et al, Molecular Cloning: A Laboratory Manual, 2d ed., Cold Spring Harbor Laboratory Press, 1989; Kunkle, PNAS 82:488, 1985). In addition, within certain embodiments of the invention, portions of the retroviral gene delivery vehicles may be derived from different retroviruses. For example, within one embodiment of the invention, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

Within one aspect of the present invention, retrovector constructs are provided comprising a 5′ LTR, a tRNA binding site, a packaging signal, one or more heterologous sequences, an origin of second strand DNA synthesis and a 3′ LTR, wherein the vector construct lacks gag/pol or env coding sequences.

Within certain embodiments of the invention, retrovirus vectors are provided wherein viral promoters, preferably CMV or SV40 promoters and/or enhancers are utilized to drive expression of one or more genes of interest.

Within other aspects of the invention, retrovirus vectors are provided wherein tissue-specific promoters are utilized to drive expression of one or more genes of interest.

Retrovirus vector constructs for use with the subject invention may be generated such that more than one gene of interest is expressed and preferably secreted. This may be accomplished through the use of di- or oligo-cistronic cassettes (e.g., where the coding regions are separated by 120 nucleotides or less, see generally Levin et al., Gene 108:167-174, 1991), or through the use of Internal Ribosome Entry Sites (“IRES”).

Within one aspect of the invention, self-inactivating (SIN) vectors are made by deleting promoter and enhancer elements in the U3 region of the 3′LTR, including the TATA box and binding sites for one or more transcription factors. The deletion is transferred to the 5′LTR after reverse transcription and integration in transduced cells. This results in the transcriptional inactivation of the LTR in the provirus. Possible advantages of SIN vectors include increased safety of the gene delivery system as well as the potential to reduce promoter interference between the LTR and the internal promoter which may result in increased expression of the gene of interest. Furthermore, it is reasonable to expect tighter control of regulatable gene therapy vectors due to the lack of an upstream promoter element in the 5′LTR.

FIV vectors are particularly preferred for use herein. FIV vectors may be readily constructed from a wide variety of FIV strains. Representative examples of FIV strains and molecular clones of such isolates include the Petaluma strain and its molecular clones FIV34TF10 and FIV14 (Olmsted et al., PNAS 86:8088-8092, 1989; Olmsted et al., PNAS 86:2448-2452, 1989; Talbot et al., PNAS 86:5743-5747, 1989), the San Diego strain and its molecular clone PPR (Phillips et al., J. Virology 64:4605-4613, 1990), the Japanese strains and their molecular clones FTM191CG and FIV-TM2 (Miyazawa et al., J. Virology 65:1572-1577, 1991) and the Amsterdam strain and its molecular clone 19K1 (Siebelink et al., J. Virology 66:1091-1097, 1992). Such FIV strains may either be obtained from feline isolates, or more preferably, from depositories or collections such as the ATCC, or isolated from known sources using commonly available techniques.

Any of the above FIV strains may be readily utilized in order to assemble or construct FIV gene delivery vehicles given the disclosure provided herein, and standard recombinant techniques (e.g., Sambrook et al., Molecular Cloning: A laboratory Manual, 2nd ed., Cold Spring Harbor Laboratory Press, 1989; Kunkle, PNAS 82:488, 1985; International Publication Nos. WO 99/15641 and WO 99/36511). In addition, within certain embodiments of the invention, portions of the FIV gene delivery vehicles may be derived from different viruses. For example, within one embodiment of the invention, recombinant FIV vector or LTR sequences may be partially derived or obtained from HIV, a packaging signal from SIV, and an origin of second strand synthesis from HIV-2.

Within one aspect of the present invention, FIV vector constructs are provided comprising a 5′ FIV LTR, a tRNA binding site, a packaging signal, one or more heterologous sequences, an origin of second strand DNA synthesis, an RNA export element and a 3′ FIV LTR. Briefly, Long Terminal Repeats (“LTRs”) are subdivided into three elements, designated U5, R and U3. These elements contain a variety of signals which are responsible for the biological activity of a retrovirus, including for example, promoter and enhancer elements which are located within U3. LTRs may be readily identified in the provirus (integrated DNA form) due to their precise duplication at either end of the genome. For purposes of the present invention, a 5′ FIV LTR should be understood to include as much of the native 5′ FIV LTR in order to function as a 5′ promoter or promoter/enhancer element to allow reverse transcription and integration of the DNA form of the vector. The 3′ FIV LTR should be understood to include as much of the 3′ FIV LTR to function as a polyadenylation signal to allow reverse transcription and integration of the DNA form of the vector.

Additionally, FIV vector constructs may contain hybrid FIV LTRs where up to 75% of the wildtype FIV LTR sequence is deleted and replaced by one or more viral or non-viral promoter or promoter/enhancer elements (e.g., other retroviral LTRs and/or non-retroviral promoters or promoter/enhancers such as the CMV promoter/enhancer or the SV40 promoter) similar to the hybrid LTRs described by Chang, et al., J. Virology 67, 743-752, 1993; Finer, et al., Blood 83, 43-50, 1994 and Robinson, et al., Gene Therapy 2, 269-278, 1995.

The tRNA binding site and origin of second strand DNA synthesis are also important for a retrovirus to be biologically active, and may be readily identified by one of skill in the art. For example, tRNA binds to a retroviral tRNA binding site by WatsonCrick base pairing, and is carried with the retrovirus genome into a viral particle. The tRNA is then utilized as a primer for DNA synthesis by reverse transcriptase. The tRNA binding site may be readily identified based upon its location just downstream from the 5′ LTR. Similarly, the origin of second strand DNA synthesis is, as its name implies, important for the second strand DNA synthesis of a retrovirus. This region, which is also referred to as the poly-purine tract, is located just upstream of the 3′ LTR.

The packaging signal sequence of FIV directs packaging of viral genetic material into the viral particle. A major part of the packaging signal in FIV lies between the 5′ FIV LTR and the gag/pol sequence with the packaging signal likely overlapping in part with the 5′ area of the gag/pol sequence.

In addition to 5′ and 3′ FIV LTRs, a tRNA binding site, a packaging signal, and an origin of second strand DNA synthesis, certain preferred recombinant FIV vector constructs for use herein also comprise one or more genes of interest, each of which is discussed in more detail below. In addition, the FIV vectors may, but need not, include an RNA export element (also variously referred to as RNA transport, nuclear transport or nuclear export elements) which may be the FIV RRE (Rev-responsive element) or a heterologous transport element. Representative examples of suitable heterologous RNA export elements include the Mason-Pfizer monkey virus constitutive transport element, the MPMV CTE (Bray et al., PNAS USA 91, 1256-1260, 1994), the Hepatitis B Virus posttranscriptional regulatory element, the HBV PRE (Huang et al., Mol. Cell. Biol. 13:7476-7486, 1993 and Huang et al., J. Virology 68:3193-3199, 1994), other lentiviral Rev-responsive elements (Daly et al., Nature 342:816-819, 1989 and Zapp et al., Nature 342:714-716, 1989) or the PRE element from the woodchuck hepatitis virus. Further RNA export elements include the element in Rous sarcoma virus (Ogert et al., J. Virology 70:3834-3843, 1996; Liu & Mertz, Genes & Dev. 9:1766-1789, 1995) and the element in the genome of simian retrovirus type 1 (Zolotukhin et al., J. Virology 68:7944-7952, 1994). Other potential elements include the elements in the histone gene (Kedes, Annu. Rev. Biochem. 48:837-870, 1970), the a interferon gene (Nagata et al., Nature 287:401-408, 1980), the β-adrenergic receptor gene (Koilka et al., Nature 329:75-79, 1987), and the c-Jun gene (Hattorie et al., PNAS 85:9148-9152, 1988).

FIV vector constructs which lack both gag/pol and env coding sequences may be used with the present invention. As utilized herein, the phrase “lacks gag/pol or env coding sequences” should be understood to mean that the FIV vector contains less than 20, preferably less than 15, more preferably less than 10, and most preferably less than 8 consecutive nucleotides which are found in gag/pol or env genes, and in particular, within gag/pol or env expression cassettes that are used to construct packaging cell lines for the FIV vector construct. This aspect of the invention provides for FIV vectors having a low probability of undesirable recombination with gag/pol or env sequences which may occur in a host cell or be introduced therein, for example, by transformation with an expression cassette. The production of FIV vector constructs lacking gag/pol or env sequences may be accomplished by partially eliminating the packaging signal and/or the use of a modified or heterologous packaging signal. Within other embodiments of the invention, FIV vector constructs are provided wherein a portion of the packaging signal that may extend into, or overlap with, the FIV gag/pol sequence is modified (e.g., deleted, truncated or bases exchanged). Within other aspects of the invention, FIV vector constructs are provided which include the packaging signal that may extend beyond the start of the gag/pol gene. Within certain embodiments, the packaging signal that may extend beyond the start of the gag/pol gene is modified in order to contain one, two or more stop codons within the gag/pol reading frame. Most preferably, one of the stop codons eliminates the gag/pol start site. In other embodiments, the introduced mutation may cause a frame shift in the gag/pol coding region.

Other retroviral gene delivery vehicles may likewise be utilized within the context of the present invention, including for example those described in EP 0,415,731; WO 90/07936; WO 91/0285, WO 9403622; WO 9325698; WO 9325234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Packaging cell lines suitable for use with the above described retrovector constructs may be readily prepared (see, e.g., U.S. Pat. Nos. 5,591,624 and 6,013,517, incorporated herein by reference in their entireties; and International Publication No. WO 95/30763), and utilized to create producer cell lines (also termed vector cell lines or “VCLs”) for the production of recombinant vector particles. Briefly, the parent cell line from which the packaging cell line is derived can be selected from a wide variety of mammalian cell lines, including for example, human cells, monkey cells, feline cells, dog cells, mouse cells, and the like.

For example, potential packaging cell line candidates are screened by isolating the human placental alkaline phosphatase (PLAP) gene from the N2-derived retroviral vector pBAAP, and inserting the gene into the FIV vector construct. To generate infectious virus, the construct is co-transfected with a VSV-G encoding expression cassette (e.g., pMLP-G as described by Emi et al., J. Virology 65, 1202-1207, 1991; or pCMV-G, see U.S. Pat. No. 5,670,354) into 293T cells, and the virus harvested 48 hours after transfection. The resulting virus can be utilized to infect candidate host cells which are subsequently FACS-analyzed using antibodies specific for PLAP. Candidate host cells include, e.g., human cells such as HeLa (ATCC CCL 2.1), HT-1080 (ATCC CCL 121), 293 (ATCC CRL 1573), Jurkat (ATCC TIB 153), supT1 (NIH AIDS Research and Reference reagent program catalog #11100), and CEM (ATCC CCL 119) or feline cells such as CrFK (ATCC CCL 94), G355-5 (Ellen et al., Virology 187:165-177, 1992), MYA-1 (Dahl et al., J. Virology 61:1602-1608, 1987) or 3201-B (Ellen et al., Virology 187:165-177, 1992). Production of p24 and reverse transcriptase can also be analyzed in the assessment of suitable packaging cell lines.

After selection of a suitable host cell for the generation of a packaging cell line, one or more expression cassettes are introduced into the cell line in order to complement or supply in trans components of the vector which have been deleted (see, e.g., U.S. Pat. Nos. 5,591,624 and 6,013,517, incorporated herein by reference in their entireties; and International Publication No. WO 95/30763). For example, packaging expression cassettes may encode either gag/pol sequences alone, gag/pol sequences and one or more of vif, rev or ORF 2, or one or more of vif, rev or ORF 2 alone and may contain an RNA export element. For example, the packaging cell line may contain only ORF 2, vif, or rev alone, ORF 2 and vif, ORF 2 and rev, vif and rev or all three of ORF 2, vif and rev.

Packaging cell lines may also comprise a promoter and a sequence encoding ORF 2, vif, rev, or an envelope (e.g., VSV-G), wherein the promoter is operably linked to the sequence encoding ORF 2, vif, rev, or the envelope. For packaging cell lines containing inducible gag/pol or env expression cassettes, additional expression cassettes facilitating the transactivation of the inducible promoter may be incorporated.

The expression cassette may or may not be stably integrated. The packaging cell line, upon introduction of an FIV vector, may produce particles at a concentration of greater than 10³, 10⁴, 10⁵, 10⁶, 10⁷, 10⁸, or, 10⁹ cfu/ml.

Preferably, lentiviral vector particles are constructed to provide for replacement of both the defective gene product and cells. For gene product replacement using neural progenitors, it is preferable that the product is secreted and is able to rescue surrounding cells. For cell replacement, progenitors should generally maintain migratory potential and integrate appropriately into degenerating regions. The use of FIV-based vectors for infection of neural progenitor cells, without significant effect on self-renewal, allows stable expression of therapeutic transgenes in progenitor cells. Lentiviral vector particles may also be used to introduce genes for enhanced cell type-specific differentiation or migration in the degenerating brain.

B. Treatment of CNS and Brain Diseases

In humans, there are numerous inherited metabolic diseases affecting the CNS, many of which result in cerebellar degeneration with concomitant symptoms such as dysmetria, ataxia, past pointing, dysdiadochokinesia, dysarthria, intention and action tremor, cerebellar nystagmus, rebound, hypotonia, and loss of equilibrium. These diseases may be alleviated using lentiviral-based, and in particular, FIV-based gene therapy as disclosed herein.

Diseases of the CNS and brain in humans that are amenable to treatment using the methods of the invention include a wide variety of diseases and disorders, including for example, spinocerebellar ataxias (SCA) such as SCA-1, SCA-2, SCA-3, SCA-6, and SCA-7; cerebellar degeneration due to alcoholism; idiopathic Purkinje cell degeneration; lithium intoxication; ceroid lipofuscinosis; ataxia telangiectasia; high dose arabinoside; Huntington's disease; fragile X syndrome; hereditary motor and sensory neuropathy and cerebellar atrophy; Alzheimer's disease (both sporadic and familial); normal aging; Parkinson's Disease and Parkinson's disease-like symptoms such as muscle tremors, muscle weakness, rigidity, bradykinesia, alterations in posture and equilibrium and dementia; demyelinating diseases such as, but not limited to, multiple sclerosis, parainfectious disorders such as acute disseminated encephalomyelitis and acute hemorrhagic leukoencephalopathy, viral infections such as progressive multifocal leukoencephalopathy and subacute sclerosing panencephalitis, nutritional disorders such as vitamin B₁₂ deficiency, demyelination of the corpus callosum (Marchiafava-Bignami disease) and central pontine myelinolysis, anoxic-ischemic sequelae such as delayed postanoxic cerebral demyelination and progressive subcortical ischemic encephalopathy; dismyelinating diseases such as, but not limited to, the leukocystrophies such as metachromatic leukodystropy, sudanophilic (Pelizaeus-Merzbacher disease), globoid cell (Krabbe's disease), adrenoleukodystropy (Schilder's disease), Alexander's disease, Canavan's disease, Seitelberger's disease, aminoaciduias; multisystem atrophy; paraneoplastic Purkinje cell degeneration; metachromatic leukodystrophy (enzymedeficient and activator-deficient form); manic depression; bipolar disorders; schizophrenia; autism; and the like.

Accordingly, the methods of the present invention may be used to alleviate abnormalities of the CNS and cerebellum that result in demyelination, dysmyelination, dementia, dysmetria, ataxia, past pointing, dysdiadochokinesia, dysarthria, intention and action tremor, cerebellar nystagmus, rebound, hypotonia, and loss of equilibrium.

Genes encoding a wide variety of polypeptides, proteins or enzymes may be employed, including those which, when expressed, prevent or alleviate the effects of the particular CNS and/or cerebellar disorder in question. Examples of such proteins include, but are not limited to CLN2 (tripeptidyl protease; ttp); CLN3; CLN1 (protein palmitoyl thioesterase); calbindin; glutamate decarboxylase; the genes encoding proteins deficient in SCA-1, SCA-2, SCA-3, SCA-6, and SCA-7; ataxin (1-7); arylsulfatase A, sulfatide activator/saposin; galactosylceramidase; various growth factors such as any of the various NGFs and FGFs, as well as CNTF, BDNF, GDNF, NT3, NT4/5, and IGF-1; monoamine oxidase; tyrosine hydroxylase; the Huntington (htt) gene; bipolar genes such as G-protein alpha subunit gene and Galphaz (GNAZ); serotonin transporter gene; serotonin receptor HTR-7, genes in the VCSF region of chromosome 22; and antiapoptotic genes. Moreover, critical enzymes involved in the synthesis of neurotransmitters such as dopamine, norepinephrine, and GABA have been cloned and available and can be used to treat a broad range of brain disease in which disturbed neurotransmitter function plays a crucial role, such as schizophrenia, manic-depressive illnesses and Parkinson's Disease. It is well established that patients with Parkinson's suffer from progressively disabled motor control due to the lack of dopamine synthesis within the basal ganglia. The rate limiting step for dopamine synthesis is the conversion of tyrosine to L-DOPA by the enzyme, tyrosine hydroxylase. L-DOPA is then converted to dopamine by the ubiquitous enzyme, DOPA decarboxylase. Thus, the genes for tyrosine hydroxylase and DOPA decarboxylase can be delivered by the techniques described herein in order to treat such diseases as Parkinson's. In addition, the enzymes responsible for neurotransmitter synthesis can be delivered using the systems described herein. For example, the gene for choline acetyl transferase may be expressed within the brain cells (neurons or glial) of specific areas to increase acetylcholine levels and improve brain function. For treating multiple sclerosis, the genes encoding MPIF-1, MIP-4 and M-CIF can be delivered (see, U.S. Pat. No. 6,001,606).

The methods of the invention also have use in the veterinary field including treatment of domestic pets and farm animals.

As utilized herein, the terms “treated, prevented, or, inhibited” refer to the alteration of a disease course or progress in a statistically significant manner. Determination of whether a disease course has been altered may be readily assessed in a variety of model systems and by using standard assays, known in the art, which analyze the ability of a gene delivery vector to delay or prevent CNS or cerebellar degeneration.

1. Methods of Administration

Gene delivery vectors may be delivered directly to the CNS or brain by injection into, e.g., a ventricle, a cerebellar lobule and/or the striatum, using a needle, catheter or related device. In particular, within certain embodiments of the invention, one or more dosages may be administered directly in the indicated manner at dosages greater than or equal to 10³, 10⁴, 10⁵, 10⁶, 10⁷, 10⁸, 10⁹, 10¹⁰ or 10¹¹ cfu. Cerebellar injections are complicated by the fact that stereotaxic coordinates cannot be used to precisely target the site of an injection; there is animal to animal variation in the size of cerebellar lobules, as well as their absolute three-dimensional orientation. Thus, cholera toxin subunit b (CTb) may be used to determine the exact location of the injection and reveal the pool of transducable neurons at an injection site. Injections may fill the molecular layer, Purkinje cell layer, granule cell layer and white matter of the arbor vitae but do not extend to the deep cerebellar nuclei.

Alternatively, and preferably for treating diseases using transduced neural progenitor cells, neural progenitor cells are first transduced ex vivo and then delivered to the CNS. Generally, if transduced ex vivo, cells will be infected with the viral vectors described herein at an MOI of about 0.01 to about 50, preferably about 0.05 to about 30, and most preferably about 0.1 to about 20 MOI. For FIV vectors, an MOI of about 0.05 to about 10, preferably about 0.1 to about 5, or even 0.1 to about 1, should be sufficient.

Once transfected ex vivo, cells can be delivered, for example, to the ventricular region, as well as to the striatum, spinal cord and neuromuscular junction, using neurosurgical techniques known in the art, and as described in the examples below, such as by stereotactic injection and injections into the eyes and ears (see, e.g., Stein et al., J. Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In general, the amount of transduced cells in the compositions to be delivered to the subject will be from about 10¹ to about 10¹⁰ cells or more, more preferably about 101 to 10⁸ cells or more, and even more preferably about 10² to about 10⁴ cells, or more. Other effective dosages can be readily established by one of ordinary skill in the art through routine trials establishing dose response curves.

2. Assays

A wide variety of assays may be utilized in order to detennine appropriate dosages for administration, or to assess the ability of a gene delivery vector to treat or prevent a particular disease. Certain of these assays are discussed in more detail below.

For example, the ability of particular vectors to transduce cerebellar neurons and neural progenitor cells can be assessed using reporter genes, as discussed below. The ability of the transduced progenitor cells to differentiate may be tested, for example, using immunocytochemistry, as discussed below in the examples.

3. Neurological Function

In mice, neurological function can be measured by EEG. Behavioral, memory, and cognitive function can be assayed using techniques known in the art. See, e.g., Chang et al., Neuro Report 4:507-510, 1993.

4. Neural Tissue Analysis

Tissues can be harvested from treated mice or primates, and processed for evaluation of neuronal degeneration, regeneration and differentiation using routine procedures. In this invention it is useful to evaluate, for example, various cerebellar neuronal tissues, including cells in the molecular layer such as Purkinje cells, stellate and basket neurons, as well as cells in the granule layer, such as fusiform Golgi neurons and granule cell neurons. Measurements performed over time can indicate increasing correction of cells distant to the vector administration site. CSF can also be collected and evaluated for protein levels or enzyme activity, particularly if the vector encodes a secretable enzyme.

5. Pharmaceutical Compositions

Gene delivery vectors may be prepared as a pharmaceutical product suitable for direct administration. Within preferred embodiments, the vector should be admixed with a pharmaceutically acceptable excipients or vehicles, and optionally other therapeutic and/or prophylactic ingredients. Such excipients include liquids such as water, saline, phosphate buffered saline, glycerol, polyethyleneglycol, hyaluronic acid, ethanol, etc. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

The delivery vectors may be provided in kits with suitable instructions and other necessary reagents, in order to transduce cells as described above. The kits can also contain, depending on the particular delivery vector used, suitable packaged reagents and materials (i.e. pharmaceutical excipients, catheters and the like).

### Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

Restriction and modifying enzymes, as well as other reagents for DNA manipulations were purchased from commercial sources, and used according to the manufacturers' directions. In the cloning of DNA fragments, except where noted, all DNA manipulations were done according to standard procedures. See, e.g., Sambrook et al., supra.

### EXAMPLE 1

### Production of Recombinant FIV Vector Particles for use in Transducing Cerebellar Cells

FIV vectors expressing E. coli β-galactosidase were generated which were devoid of vif and ORF 2 (FIVβglucΔvifΔorf2), were generated essentially as described in International Publication No. WO 99/36511, published Jul. 22, 1999. Specifically, FIV packaging constructs were generated in a series of steps from the full-length FIV molecular clone, FIV-34TF10 (NIH AIDS Research and Reference Reagent Program, Cat. No. 1236; Phillips et al., J. Virol. 66: 5464, 1992, Talbott et al., PNAS 86: 5743, 1989) as described (Johnston et al., J. Virol 73:4991-5000, 1999). The FIV vector construct, pVET_{L}Cβgal (PVET_{L}Cβ in ref (Johnston et al., J. Virol 73:4991-5000, 1999), was generated by inserting an expression cassette consisting of the CMV promoter followed by the β-galactosidase gene into the PVET_{L} FIV vector backbone. The PVET_{L} backbone contains the FIV 5′ LTR, in which the FIV U3 region is replaced by the CMV promoter/enhancer, 0.5 kb of Gag coding region, a multicloning site and the FIV 3′ LTR (Johnston et al., J. Virol 73:4991-5000, 1999). All constructs were screened by restriction enzyme digestion and the sequence of regions amplified by PCR confirmed by sequence analysis. Oligonucleotides were synthesized by Operon Technologies, Inc.(Alameda, Calif.) and sequences as well as more detailed cloning methods are available upon request.

Construction of the VSV-G envelope expression plasmid, pCMV-G, has been described (Yee et al., PNAS 91:9564-9568, 1994). Pseudotyped FIVβgal vector particles were generated by transient transfection of plasmid DNA into 293T cells plated one day prior to transfection at a density of 2.8×10⁶ cells per 10 cm diameter culture dish. Cotransfections were performed using a 1:2:1 molar ratio of FIV packaging construct, FIV vector construct and VSV-G envelope-expressing plasmid. DNA complexes were prepared using calcium phosphate (Profectin kit; Promega Corp. Madison, Wis.) and transfected into cells according to the manufacturer's instructions. The medium was replaced 8-16 hr after transfection and the supernatant harvested twice between 32 and 48 hr after the start of transfection. The harvested supernatants were filtered through a 0.45 M Nalgene filter and stored at −70° C. or concentrated prior to storage. Supernatants were concentrated by centrifugation (Johnston et al., J. Virol 73:4991-5000). Vector titers were determined on HT1080 cells by serial dilution and assay for β-galactosidase (Li et al., PNAS 92:7700-7704, 1995). rFIV titers were approximately 5-10×10⁷ infectious units/ml.

In some experiments, a neuronal tracer, cholera toxin subunit b (CTb, List Laboratories, California), was added at 1 μg/μl to the viral suspension. CTb is the nontoxic subunit of cholera toxin and has previously been used to define the limits of an injection site in experiments with pseudorabies virus (Chen et al., Brain Research 838:171-183, 1999). In this study, CTb immunoreactivity allowed independent visualization of cerebellar injection sites. In this manner, transport and spread of virus outside of the injection site could be distinguished from transduction within the primary injection site.

### EXAMPLE 2

### Use of Recombinant FIV Vector Particles to Transduce Cerebellar Cells

Young adult C57BL/6 mice weighing 20-25 g were anesthetized with ketamine/xylazine. A burr hole was drilled at the midline posterior occipital bone overlying the cerebellar anterior lobe. Pressure injections (2 μl total) were made into a single cerebellar lobule using a Hamilton syringe cemented with a glass micropipette tip. A total of 16 animals were injected with rFIVβgal; 8 with rFIVβgal alone and 8 with rFIVβgal plus CTb. Animals were sacrificed at 3, 6 (rFIVβgal) or 7 (rAAVβgal) weeks after gene transfer and cerebella, brainstems and thoracolumbar spinal cords removed. Tissues were postfixed in 4% paraformaldehyde overnight at 4° C., cryoprotected for 1-3 days in 30% sucrose in phosphate buffered saline at 4° C. and then sectioned on a cryostat at 50 μm thickness (cerebellum/brainstem sagitally and spinal cord longitudinally). All sections were collected and stored at −20° C. until use.

For histochemistry and immunofluorescence, every other section was processed for β-galactosidase activity using 5-bromo-4-chloro-4-indolyl β-D-galactoside (X-Gal) according to Davidson et al. (Davidson et al., PNAS 97:3428-3432, 2000). Transport and spread of virus was determined by comparing the X-gal processed sections to adjacent sections that had been processed for CTb immunohistochemistry. CTb immunochemistry was done according to Alisky and Tolbert, J. Neurosci 52:143-148, 1994. Briefly, sections were blocked overnight in 2% rabbit serum in Tris-buffered saline, followed by 24 hours in goat anti-choleragenoid (List Laboratories) diluted 1/10,000. Sections were then incubated with biotinylated, rabbit anti-goat secondary antibodies and processed using an avidin-biotin peroxidase substrate. Neuronal versus glial transduction was determined by dual immunofluoresence for β-galactosidase and neuronal (calbindin) or glial (glial fibrillary acidic protein [GFAP]) markers on free-floating sections. Both anticalbindin and anti-GFAP were purchased from Sigma Biochemicals (St. Louis, Mo.) and were used at a concentration of 1/3,000 or 1/1000, respectively. Immunofluorescence was evaluated using a confocal microscope and associated software.

β-galactosidase-expressing Purkinje cells were counted in every other 50 μm cerebellum section under a 10× brightfield objective. Purkinje cells were selected for quantitation because they can be quickly counted in thicker sections without stereological correction.

Cerebellar injections are complicated by the fact that stereotaxic coordinates cannot be used to precisely target the site of an injection; there is animal to animal variation in the size of cerebellar lobules, as well as their absolute three-dimensional orientation. Cholera toxin subunit b (CTb) was therefore used. CTb is a reagent commonly used to track neurons from their termini or projections to their somata. The use of Ctb allowed two advantages: First, the exact location of the injection could be determined. Second, it revealed the pool of potentially transducable neurons at an injection site. In mice injected with virus plus CTb, CTb-immunoreactivity was found encompassing the targeted cerebellar lobule. In some cases, injections encompassed the dorsal half of one lobule and the ventral half of another lobule, while in other cases only a portion of a lobule was injected. At most, injections filled the molecular layer, Purkinje cell layer, granule cell layer and white matter of the arbor vitae but never extended to the deep cerebellar nuclei. Outside the injection site, the CTb retrogradely labeled precerebellar neurons in the cuneate, vestibular, olivary, reticular and spinal nuclei. Thus CTb co-injections mapped an extensive pool of neurons which could be potentially transduced via retrograde axonal transport of recombinant virus.

Histochemistry for β-galactosidase indicated that rFIVβgal mediated transduction to large numbers of neurons. The cytoplasmically targeted β-galactosidase allowed detection of the Purkinje cell dendritic arbors, and also somata and their axonal extensions to the deep cerebellar and vestibular nuclei. The number of Purkinje cells transduced ranged from 78 to 1575 or 230 to 1298 in the rFIVβgal or rFIVβgal plus CTb-injected mice, respectively. Thus, CTb had no significant effect on rFIV-mediated gene transfer, either positively or negatively. The number of transduced Purkinje cells was proportional to the area positively labeled with CTb. For example, in cerebella with fewer CTb-labeled cells, there were fewer β-galactosidase-positive cells. Also, transduction was generally confined to the CTb-positive region. However, in some cerebella Purkinje cells in lobules adjacent to the injection site were also transduced.

In addition to Purkinje cells rFIVβgal transduced stellate and basket neurons in the molecular layer. In the granule cell layer, large numbers of fusiform Golgi neurons were transduced, but only scattered granule cell neurons. Retrograde transport of rFIVβgal was limited to deep cerebellar nuclei and lateral vestibular nuclei, which are the nuclei physically closest to the injection sites. Neurons in the cuneate, reticular, olivary and lumbar spinal nuclei were positive for CTb in rFIVβgal, CTb co-injected animals. However, these neurons were never β-galactosidase positive, indicating that rFIV has a limited ability to be retrogradely transported. In all cases, FIVβgal transduction was exclusively neuronal, with no co-localization of β-galactosidase with the glial marker GFAP.

To the best of the inventors' knowledge, this is the first report of direct gene transfer to cerebellar Purkinje cells. The above findings in the cerebellum are consistent with earlier results demonstrating transduction of cerebral neurons with recombinant lentivirus or AAV vectors. In addition the above data revealed selectivity among potential-target neurons, a previously unknown characteristic of rFIV vectors. This observation was likely made as a consequence of the morphologically distinct classes of neurons in each layer of the cerebellar cortex. Selective tropism could be more difficult to detect upon gene transfer to the basal ganglia, because lamination and neuronal subtypes are more complex.

Within the molecular layer of a cerebellar lobule, stellate cells are located towards the outside while basket neurons are nearer the Purkinje cell bodies. The gigantic Purkinje cells form a monolayer, while fusiform Golgi neurons and small granule neurons are exclusively in the granule cell layer. The above data show that rFIV-based vectors transduce neurons in the molecular and Purkinje cell layer, with limited transduction of Golgi neurons and almost no gene transfer to granule neurons. These data suggest that rFIV vectors are useful as therapies for diseases in which Purkinje cells degenerate.

The anterior lobe of the cerebellum receives input from multiple nuclei as well as the cervical and lumbar spinal cord segments. As such, injections into the cerebellum allowed for direct evaluation of the ability of rFIV-based vectors to undergo retrograde transport. Axonal transport with rFIV-based vectors was limited to the spatially closest nuclei.

Further studies with thin sections (10-20μ) and stereological sampling are done in order to quantify transduction of smaller neurons such as Golgi, stellate and basket cells and also to detect the small numbers of granule cells that are transduced. In the above study, X-gal reaction product filled the Purkinje cell dendrites of the molecular layer, making visualization of stellate and basket cells difficult. A two microliter injection (10⁴-10⁵ infectious units) into a single lobule transduced up to 1500 Purkinj e cells. With an estimated 20,000 Purkinje cells in all 10 lobules of the mouse cerebellum (Caddy and Biscoe, Brain Research 111:306-398, 1976) approximately 10% of all Purkinje cells and close to 100% of the injected lobule were transduced. With injections into multiple lobules it is reasonable to assume that most or all Purkinje cells could ultimately be transduced with rFIV-based vectors. Thus, these vectors are useful in discerning the underlying mechanisms of degeneration in diseases where Purkinje cells degenerate, such as the human disorders SCA-2 or SCA-6, or the murine cerebellar degeneration models.

Thus, FIV efficiently transfects Purkinje cells and other cortical neurons and shows promise in correction of cerebellar degeneration both hereditary and acquired.

### EXAMPLE 3

### Transduction of Neural Progenitor Cells

### Using Viral Vectors

In order to test the ability of various viral vectors to transduce neural progenitor cells the following materials and methods were used.

Neurosphere Generation and Differentiation

Progenitor cells from embryonic (day 15-17) mice were obtained essentially as described (Reynolds et al., J. Neurosci. 12:4565-4574, 1992). Briefly, embryonic brain was cleared of meninges, diced with a scalpel blade and triturated in Hibernate media (GIBCO, MD) containing 6 g/L total glucose and B27 supplement (GIBCO, MD). Single cells (1×10⁵ cells/mL) were cultured in DMEM/F 12 containing a final glucose concentration of 6 g/L, ITS supplement (Sigma, St. Louis, Mo.), 2 mM glutamine, penicillin/streptomycin and 20 ng/mL EGF or 20 ng/mL bFGF (Sigma, St. Louis Mo.). After 4-7 days and every 4 days subsequent, neurospheres were mechanically dissociated and the media replaced.

For differentiation in vitro, neurospheres were plated onto 0.01% polyornithinecoated 24 well plates in DMEM/F12 media containing 6 g/L glucose, 2 mM glutamine, penicillin/streptomycin, 1% FBS and either B27 supplement (Brewer et al., J. Neurosci.Res. 35:567-576, 1993) or IGF-1 (20 ng/mL; Sigma, St. Louis, Mo.).

Viral Constructs

Adenoviral vectors expressing either eGFP or nuclear targeted β-galactosidase in E1 were produced using the RAPAd™.I system (Anderson et al., Gene Ther. 7:1034-1038, 2000). FIV constructs were made by cloning cytoplasmic β-galactosidase or eGFP sequences into the pVETLRmcs plasmid (Johnston et al. J. Virol. 73:4991-5000, 1999). The resulting plasmids were co-transfected with pCFIVΔorfΔvif and pCMV.G (Yee et al., PNAS. 91:9564-9568, 1994) into HT1080 cells. Viral particles were collected from the media over 4 days and centrifuged at 7500× G to concentrate particles. Viral particles were resuspended in 40 mg/mL lactose in PBS. Recombinant AAV vectors based on AAV2, 4 or 5 were prepared as previously described (Chiorini et al., J. Virol. 73:4293-4298, 1999; Chiorini et al., Hum. Gene Ther. 6:1531-1541, 1995). Adenoviral and AAV transgenes were under the control of the Rous sarcoma virus LTR promoter (RSVp). FIV constructs contained the cytomegalovirus promoter (CMV).

Viral Infection of Neurospheres

Neurospheres in EGF or bFGF-containing media (passage number 3-6) were infected with FIV (MOI 0.1-0.5) or adenovirus (MOI 20) expressing β-galactosidase or eGFP in a small volume (200 spheres/500 μL media). Samples of neurosphere preparations were dissociated to estimate cell numbers for MOI calculations. After 18 h or 1 h respectively the media was changed and 10 mL added. Expression was monitored over time in EGF-containing media, or neurospheres were differentiated 5 days post infection.

Animals and Injections

Six week old C57B1/6 mice were purchased from Jackson Laboratories (Bar Harbor, MN) and housed at the University of Iowa Animal Care Facility. All animal procedures were approved by the University of Iowa Animal Care and Use Committee. Neurospheres were infected with FIVβgal as described above at least 2 weeks prior to transfer into the mouse CNS. Neurospheres were dissociated with a single pass through a 23 gauge needle and approximately 100,000 cells were injected into the striatum (bregma +0.4 mm rostral, 2 mm lateral, at a depth of 3 mm). For tumor-induced migration studies, animals were injected with 100,000 rat C6 tumor cells (ATCC, Manassas, Va.) either 15 weeks after or 3 days prior to progenitor cell injection to create an acute injury model (Benedetti et al., Nat.Med. 6:447-450, 2000; Herrlinger et al., Mol.Ther. 1:347-357). Ten days, 10 weeks, or 15 weeks after injection, animals were perfused with 2% paraformaldehyde and processed as previously described (Ghodsi et al., Hum. Gene Ther. 9:2331-2340, 1998) for X gal histochemistry or immunocytochemistry.

Immunocytochemistry

For cell-type analysis the following antibodies were used; mouse monoclonal glial fibrillary acidic protein (GFAP; 1:3000) conjugated to Cy3, mouse monoclonal MAP2 (1:250;), CNPase (1:1000; Sigma Immunochemicals, St Louis, Mo.) and rabbit polylclonal β-galactosidase (1:1500; BioDesign International, Saco, MN). Rat 401 which recognizes the progenitor marker, nestin, was obtained from the Developmental Hybridoma Bank, University of Iowa and used at 1:5. Secondary antibodies were goat anti-rabbit or mouse Alexa 488 (Molecular Probes, Eugene, Oreg.), or goat anti-mouse Rhodamine X (Jackson ImmunoResearch, West Grove, Pa.).

All antibodies were diluted in 3% BSA, 0.1% saponin, 0.3% Triton X-100 in PBS (diluent). After blocking tissues or cells for 30 minutes in diluent containing 10% goat serum, samples were incubated with primary antibodies overnight at 4° C. Sections were washed extensively in diluent and stained with fluorescently-labeled secondary antibodies for 2 h at room temperature. Photomicrographs were captured using Adobe Photoshop and a SPOT/RT digital camera (Diagnostic Inst. Sterling Heights, Mich.) on a Leicz DMRBE upright microscope or Olympus IX70 inverted microscope. For colocalization studies, samples were analyzed on a Zeiss LSM confocal microscope and associated software.

### EXAMPLE 3A

### Neurosphere Generation

Neural progenitors were isolated from embryonic day 15-17 mouse brain and maintained in EGF or bFGF-containing media as described above. Earlier work showed that cells propagated in EGF or bFGF have stem cell characteristics, are self-renewing and are multipotent. Over 7-10 days, single cells formed neurospheres in suspension. Cells within the neurospheres expressed the progenitor cell marker, neuroepithelial stem cell intermediate filament (“nestin”).

Neurospheres differentiated in vitro into neurons, astrocytes and oligodendrocytes with removal of the mitogen and addition of a substrate as previously described (Reynolds et al. Science 255:1707-1710, 1992). In contrast to neurospheres differentiated in the presence of growth factors such as IGF-1 or BDNF, spheres differentiated in 1% serum plus B27 supplement had reduced astrocyte migration without similar effect on neurons. In B27 supplemented media neurons migrated beyond the glial border similarly to neurons generated by culture in BDNF or IGF-1.

### EXAMPLE 3B

### Ability of Adenoviral Vectors to Induce Astrocyte Differentiation Within Neurospheres

In order to test the ability of adenoviral vectors to transduce neurospheres and induce astrocyte differentiation, the following experiment was conducted. Adenoviral vectors infect glia and neurons by retrograde transport in the CNS (Davidson et al., Nat. Genet. 3:219-223, 1993; Mastrangeli et al., Clin.Res. 41:223A(Abstract), 1993; Ghadge et al., Gene Ther. 2:132-137, 1995). Recombinant adenovirus (MOI 1 or 20) was used to infect cells within neurospheres, as described above. Transgene expression was observed within 16 h of infection, using the eGFP reporter and maintained for at least 1 month (last time point tested). Within 7 days an apparent change in morphology of transduced cells was seen, concomitant with an increase in GFAP expression, indicating that adenoviral infection induced astrocytic differentiation of progenitor cells. Analysis of neurospheres or cryosections stained for nestin and GFAP confirmed the loss of nestin and gain of GFAP expression. Differentiation of Ad-infected neurospheres showed that the majority of transduced cells were immunopositive for GFAP and not MAP2.

Thus, recombinant adenovirus resulted in premature differentiation of neural progenitor cells into astrocytes, even in the presence of EGF. Glial processes became evident between 5 and 7 days after infection. Several studies have reported adenoviral infection of neural progenitor cells in monolayer culture prior to transplantation. Cortis et al., Nat Biotechnol 17:349-354, 1999, showed that human neural progenitors infected at a similar MOI to this study, could express a gene product after transplantation to the rat striatum. This study did not address the differentiation of these cells either in vivo or in vitro. Brüstle et al., Nat Biotechnol 16:1040-1044, 1998, showed that Ad-infected human neural progenitors transplanted into embryos 24 h after infection could differentiate into neurons. Similarly, Gage et al., PNAS. 92:11879-11883, 1995 showed in vivo differentiation of Ad-infected rat progenitor cells transplanted into the adult rat hippocampus. Again transplants were performed 24 h post infection. These results combined with the current study suggest that adenovirus can infect neural progenitor cells. Over time in culture, infection induces differentiation. The presence of adenoviral genes especially those remaining in E4 such as orf 6 may induce differentiation by inhibiting cell division (Goodrum et al, J. Virol. 73:7474-7488, 1999). To test this hypothesis, adenoviral constructs devoid of E4 sequences are tested for their effects on progenitor differentiation.

### EXAMPLE 3C

### Ability of AAV Vectors to Transduce Neurospheres

AAV2, 4 and 5 have previously been shown to infect differentiated cells in the CNS, predominantly neurons (McCown et al., Brain Res. 713:99-107, 1996; Sutton et al., J. Virol. 73:3649-3660, 1999; Davidson et al., PNAS 97:3428-3432, 2000; Yandava et al., PNAS 96:7029-7034, 1999). These viruses were tested for transduction of neurospheres at MOI of 2. Spheres were maintained for 6 days and differentiated or analyzed for transgene expression after 6 further days. AAV2 infected less than 0.01% of cells, while no transgene positive cells were seen after infection with AAV4 or AAV 5.

Thus, none of the AAV serotypes significantly infected any cells within the neurospheres cultured in the presence of EGF. The lack of infection may be due to viral receptor competition with media components or the lack of appropriate receptors. Alternatively, the extracellular matrix components of the neurospheres may interfere with viral uptake.

AAV2 has been shown to require heparin sulfate proteoglycan for infection and uses the fibroblast growth factor receptor and αvβ5 integrin as co-receptors (Qing et al., Nat.Med. 5:71-77, 1999; Summerford et al., Nat.Med. 5:78-82, 1999). Although the receptors for AAV4 and AAV5 are unknown they are insensitive to heparin and differentially infect several cell lines and CNS cell types (Davidson et al., PNAS 97:3428-3432, 2000; Alisky et al., NeuroReport 11:2669-2673, 2000).

### EXAMPLE 3D

### Ability of FIV Vectors to Transduce Neurospheres

In order to test the ability of FIV vectors to transduce neurospheres, the following experiment was conducted. FIVeGFP was used at an MOI of 0.1-0.5 and transgene expression monitored as described above. Expression was detectable by 24 h and persisted to at least 28 days (last time-point tested). In order to confirm that a nestin-positive, self-renewing population of progenitor cells were infected by FIV, neurospheres were dissociated into single cells prior to infecting with FIVβgal. Regeneration of neurospheres was monitored over 7 days, and cryosections stained for nestin, GFAP, MAP2 and β-galactosidase. Few glial and no neuronal profiles were noted. No qualitative difference was seen in nestin expression between infected and non-infected neurospheres. The majority of cells were nestin-positive, and spheres were either completely positive or completely negative for β-galactosidase. These results indicate that FIV did not prevent self-renewal or progenitor cell maintenance. The lack of detected mosaics (negative and positive cells with an individual sphere) confirmed the clonal derivation of the neurospheres.

To assess the effect of FIV infection on progenitor differentiation, FIV-infected neurospheres were differentiated in B27 or IGF 1-containing media, 5 days post-infection. Both astrocytes and neurons expressed β-galactosidase as seen morphologically by Xgal histochemistry. Cell types were confirmed using immunocytochemistry as described above. Dual labeling with antibodies against β-galactosidase and MAP2 or GFAP confirmed that both neurons and glia could be derived from rFIV transduced progenitor cells.

### EXAMPLE 3E

### Engraftment and Differentiation Potential of FIV-infected Neurospheres

FIVβgal infected neurospheres were tested in vivo for engraftment and differentiation potential. Neurospheres were infected and maintained in vitro for 15 days prior to transplant, to reduce the possibility of free virus being carried over into the transplanted brain and infecting host parenchyma. The spheres were dissociated and stereotactically injected into the striatum of normal C57B1/6 mice and sacrificed at 10 days or 10 or 15 weeks.

After 10 days in vivo β-galactosidase positive cells were found in the striatum with scattered cells located in the needle tract and along the corpus callosum. A similar distribution was shown at 10 and 15 weeks. While the progenitor cells apparently lost nestin expression, there was little differentiation into neurons or glia as assessed by Neu N and GFAP immunocytochemistry. Migration of cells was evaluated through the rostral caudal extent of the brain. Neurosphere-derived cells cultured in EGF or bFGF containing maintenance media were found up to 800 μm from the injection site in the rostral-caudal direction. Along the corpus callosum cells migrated up to 1 mm from the position of the needle tract.

### EXAMPLE 3F

### Lesion-induced Cell Migration

To examine the effect of rFIV-mediated gene transfer on the in vivo migratory potential of neural progenitor cells, a well established model was used (Benedetti et al., Nat.Med. 6:447-450, 2000; Aboody et al., PNAS97:12846-12851, 2000). A tumor cell line, C6, was injected into the contralateral hemisphere from which FIVβgal-infected neurospheres were implanted 15 weeks before or 3 days after. One week later the mice were sacrificed and analyzed for tumor cell growth and progenitor cell migration. The tumor cells were immunoreactive for rat IgG and negative for GFAP labeling. Reactive astrocytosis was evident around the lesion site. Sections stained for β-galactosidase showed that in the absence of a tumor, cells remained around the injection site as described above. β-galactosidase labeled progenitor cells in mice injected with tumors were no longer found in the injected hemisphere but rather in the contralateral hemisphere within the tumor mass. Thus, rFIV-transduced progenitor cells retained migratory ability with the injured CNS.

To the best of the inventors' knowledge, this is the first study describing FIVmediated gene delivery to primary neural progenitor cells. Recombinant FIV pseudotyped with the VSV-G envelope transduced nestin positive neural progenitor cells and did not affect their potential for self-renewal or differentiation into neurons or glia in vitro. These cells also retained the capacity to migrate into injured regions as described for mouse C17.2 neural cell lines (Herrlinger et al., Mol Ther. 1:347-357; Snyder et al., PNAS 94:11663-11668, 1997), a rat cell line (Benedetti et al., Nat.Med. 6:447-450, 2000), and primary mouse neurospheres transduced with a retrovirus (Benedetti et al., Nat.Med. 6:447-450, 2000). The lack of significant differentiation of FIV-infected progenitors after striatal injection is similar to previously published results where differentiation is limited in the adult striatum/corpus callosum (Gage et al., PNAS. 92:11879-11883, 1995). Any differentiation in this region is predominantly glial (Gage et al., PNAS. 92:11879-11883, 1995), unless cells are injected at a stage where neurogenesis is occurring.

Accordingly, lentiviral vectors and methods of using the same for transducing neural cells, as well as for the treatment of brain disorders have been disclosed. From the foregoing, it will be appreciated that, although specific embodiments of the invention have been described herein for purposes of illustration, various modifications may be made without deviating from the spirit and scope of the appended claims.

1. A method for transducing a neural cell selected from the group consisting of a cerebellar neuron and a neural progenitor cell, said method comprising:
(a) providing a lentiviral vector particle, wherein said vector particle is produced from a lentiviral vector comprising a 5′ lentiviral LTR, a tRNA binding site, a packaging signal, a promoter operably linked to a polynucleotide encoding a protein of interest, an origin of second strand DNA synthesis and a 3′ lentiviral LTR; and
(b) administering said lentiviral vector particle to the neural cell under conditions whereby the protein encoded by the polynucleotide is expressed to produce a transduced neural cell.

2. The method of claim 1, wherein said lentiviral vector comprises 5′ and/or 3′ LTRs from a virus selected from the group consisting of HIV, HIV-1, HIV-2, FIV and SIV.

3. The method of claim 2, wherein said lentiviral vector comprises 5′ and/or 3′ LTRs from FIV.

4. The method of claim 1, wherein said neural cell is a cerebellar neuron.

5. The method of claim 4, wherein said cerebellar neuron is a Purkinje cell.

6. The method of claim 4, wherein said cerebellar neuron is transduced in vivo in a vertebrate subject in need of treatment of a disease that causes cerebellar degeneration.

7. The method of claim 4, wherein said cerebellar neuron is transduced ex vivo and the transduced neuron is introduced into a vertebrate subject in need of treatment of a disease that causes cerebellar degeneration.

8. The method of claim 1, wherein said neural cell is a neural progenitor cell.

9. The method of claim 8, wherein said neural progenitor cell is transduced in vivo in a vertebrate subject in need of treatment of a central nervous system disorder.

10. The method of claim 8, wherein said neural progenitor cell is transduced ex vivo and the transduced cell is introduced into a vertebrate subject in need of treatment of a central nervous system disorder.

11. A method for transducing cerebellar neurons comprising:
(a) providing an FIV vector particle, wherein said vector particle is produced from an FIV vector comprising a 5′ FIV LTR, a tRNA binding site, a packaging signal, a polynucleotide encoding a protein of interest operably linked to an FIV LTR promoter or a promoter element, an origin of second strand DNA synthesis and a 3′ FIV LTR; and
(b) administering said FIV vector particle to a cerebellar neuron under conditions whereby the protein encoded by the polynucleotide is expressed to produce a transduced cerebellar neuron.

12. The method of claim 11, wherein the promoter is a CMV, RSV or SV40 promoter.

13. The method of claim 11, wherein said cerebellar neuron is transduced in vivo in a vertebrate subject in need of treatment of a disease that causes cerebellar degeneration.

14. The method of claim 11, wherein said cerebellar neuron is transduced ex vivo and the transduced cerebellar neuron is introduced into a vertebrate subject in need of treatment of a disease that causes cerebellar degeneration.

15. A method for transducing neural progenitor cells comprising:
(a) providing an FIV vector particle, wherein said vector particle is produced from an FIV vector comprising a 5′ FIV LTR, a tRNA binding site, a packaging signal, a polynucleotide encoding a protein of interest operably linked to an FIV LTR promoter or a promoter element, an origin of second strand DNA synthesis and a 3′ FIV LTR; and
(b) administering said FIV vector particle to a neural progenitor cell under conditions whereby the protein encoded by the polynucleotide is expressed to produce a transduced cell.

16. The method of claim 15, wherein the promoter is a CMV, RSV or SV40 promoter.

17. The method of claim 15, wherein said neural progenitor cell is transduced in vivo in a vertebrate subject in need of treatment of a central nervous system disorder.

18. The method of claim 15, wherein said neural progenitor cell is transduced ex vivo and the transduced cell is introduced into a vertebrate subject in need of treatment of a central nervous system disorder.

19. A method of treating or preventing cerebellar neuronal degeneration in a vertebrate subject, comprising administering to the subject a lentiviral vector particle, wherein said vector particle is produced from a lentiviral vector comprising a 5′ lentiviral LTR, a tRNA binding site, a packaging signal, a promoter operably linked to a polynucleotide encoding a protein of interest, an origin of second strand DNA synthesis and a 3′ lentiviral LTR.

20. A method of treating or preventing cerebellar neuronal degeneration in a vertebrate subject, comprising administering to Purkinje cells of the subject an FIV vector particle, wherein said vector particle is produced from an FIV vector comprising a 5′ FIV LTR, a tRNA binding site, a packaging signal, a polynucleotide encoding a protein of interest operably linked to an FIV LTR promoter or a promoter element, an origin of second strand DNA synthesis and a 3′ FIV LTR.

21. A method of treating or preventing a central nervous system disorder in a vertebrate subject, comprising administering to the subject a lentiviral vector particle, wherein said vector particle is produced from a lentiviral vector comprising a 5′ lentiviral LTR, a tRNA binding site, a packaging signal, a promoter operably linked to a polynucleotide encoding a protein of interest, an origin of second strand DNA synthesis and a 3′ lentiviral LTR.

22. A method of treating or preventing a central nervous system disorder in a vertebrate subject, comprising administering transduced neural progenitor cells intraventricularly to the subject, wherein said neural progenitor cells have been transduced with an FIV vector particle, wherein said vector particle is produced from an FIV vector comprising a 5′ FIV LTR, a tRNA binding site, a packaging signal, a polynucleotide encoding a protein of interest operably linked to an FIV LTR promoter or a promoter element, an origin of second strand DNA synthesis and a 3′ FIV LTR.
